(19) United States
(12) Patent Application Publication
(10) Pub. No.: US 2019/0201100 A1
(43) Pub. Date: Jul. 4, 2019

(54) METHOD OF REDUCING RETRO-REPULSION DURING LASER LITHOTRIPSY

(71) Applicants: JOE D. BROWN, PANAMA CITY BEACH,, FL (US); DANIEL MALPHURS, PANAMA CITY BEACH, FL (US)

(72) Inventors: JOE D. BROWN, PANAMA CITY BEACH,, FL (US); DANIEL MALPHURS, PANAMA CITY BEACH, FL (US)

(21) Appl. No.: 16/234,690

(22) Filed: Dec. 28, 2018

Related U.S. Application Data
(60) Provisional application No. 62/611,030, filed on Dec. 28, 2017.

(51) Int. Cl. A61B 18/26; A61B 1/00

(52) U.S. Cl. CPC A61B 18/26; A61B 2018/00625; A61B 1/00137; A61B 1/0008

(57) ABSTRACT

A method of reducing retro-repulsion of a kidney stone during a laser lithotripsy procedure utilizes a protective cap or sleeve fitted onto an end of the laser delivery fiber and extending a predetermined distance beyond a tip of the fiber to prevent contact between the tip of the fiber and a stone to limit retro-repulsion by maintaining contact between the protective cap and the stone, thereby limiting the amount of water in the path of the laser between the fiber tip and the stone. The method may further include the step of firing the laser only when contact between the protective cap and the stone is detected, implemented manually or by a contact detection system. The fiber tip may be outwardly tapered to collimate the laser beam and allow an increased spacing between the fiber tip and the distal end of the protective cap.

This application claims the benefit of U.S. Provisional Patent Appl. Ser. No. 62/611,030, filed Dec. 28, 2017, and incorporated herein by reference.

### BACKGROUND OF THE INVENTION

### 1. Field of the Invention

The invention relates to the field of laser surgery, and in particular to a method of reducing retro-repulsion of a kidney stone during a laser lithotripsy procedure.

The method of the invention utilizes a protective cap of the type disclosed in copending PCT Appl. Ser. No. PCT/US2017/031091 (PCT Publ. No. WO/2017/192869), filed May 4, 2017, and incorporated herein by reference, to limit retro-repulsion by maintaining contact between the protective cap and the stone, thereby limiting the amount of water in the path of the laser between the fiber tip and the stone.

The method of the invention may further utilize the step of firing the laser only when contact between the protective cap and the stone is detected, implemented for example by a contact detection system and method of the type disclosed in copending U.S. patent application Ser. No. 15/992,609 (U.S. Pat. Publ. No. 2018/0344405), filed May 30, 2018, and also incorporated herein by reference.

### 2. Description of Related Art

Laser lithotripsy is a surgical procedure to remove stones from urinary tract, i.e., kidney, ureter, bladder, or urethra, and was invented during the 1980s to remove impacted urinary stones. Early laser lithotripsy methods utilized pulsed-dye lasers with picosecond pulse durations to created cavitation bubbles that collapse and cause laser induced shockwaves with a high degree of retro-repulsion.

More recently, pulsed Holmium lasers have been developed with longer pulse durations (250 micro seconds) that produce a weaker pressure wave, and therefore less retro-repulsion, while still destroying the stones. Nevertheless, retro-repulsion continues to be a problem since it requires the fiber tip position to be frequently adjusted during a procedure, prolonging the procedure.

Copending PCT Appl. Ser. No. PCT/US2017/031091 describes various protective caps or sleeves that and placed over the end of the fiber and that serve to prevent contact between the stone and the tip of the optical fiber, substantially reducing erosion of the fiber tip and in addition providing such advantages as protection of the interior of the scope during insertion of the fiber into the scope. However, the copending PCT publication does not disclose a method of using the protective caps disclosed therein that involves intentionally maintaining contact between the protective cap and the stone in order to reduce retro-repulsion and enhance lasing efficiency.

Copending U.S. patent application Ser. No. 15/992,609 discloses a method of detecting contact between a stone and a fiber tip, for the purpose of limiting such contact, but which can also be used to detect proximity between the fiber tip and the stone. In one embodiment, a detection method is disclosed that involves analyzing a spectrum for the presence of vaporized material indicative of stone distance from the fiber tip, and which can be used to reducing surgery time by causing the laser to pulse only when the fiber tip is in an optimal position for target vaporization, thus reducing extraneous pulses that cause target retro-repulsion and wear on equipment. This detection method can, in one preferred embodiment, be combined with the method of the present invention to achieve optimal therapeutic lasing efficiency.

### SUMMARY OF THE INVENTION

The present invention provides a method of reducing retro-repulsion during a lithotripsy procedure. It may be used in connection with a pulsed Holmium laser or other types of laser lithotripsy apparatus or systems.

The method of reducing retro-repulsion involves utilizing a protective cap of the type disclosed in PCT Appl. Ser. No. PCT/US2017/031091 to prevent contact between the fiber tip and the stone, while at the same time maintaining contact between the protective cap and the stone.

The protective cap may be a soft tip as disclosed in PCT Appl. Ser. No. PCT/US2017/031091, or may be made of a harder material such as glass, ceramic, or metal.

By maintaining contact between the protective cap and the stone, the method of the invention reduces retro-repulsion by limiting the amount of water between the stone and the fiber tip. Limiting the amount of water also has the effect of improving lasing efficiency by reducing energy losses to boiling of the water between the fiber and the stone surface.

These effects may be enhanced by using lower power pulses or a continuous low power pulse to maintain a continuous air bubble within the protective cap, thereby further reducing retro-repulsion, and only firing the laser when the protective cap is in contact with the stone, for example by either using the proximity detection method and apparatus disclosed in copending U.S. patent application Ser. No. 15/992,609, cited above, or by replacing lasing control in response to proximity detection with manual control of high power therapeutic pulse delivery. Manual control can be achieved through, for example, the use of an operator manipulated foot pedal.

Still further reductions in retro-repulsion may optionally be achieved by collimating the laser output, for example by using an outwardly tapered fiber tip.

### BRIEF DESCRIPTION OF THE DRAWINGS

FIG. 1 shows various fiber tip types that may be used with the method of the present invention.

FIGS. 2A-2C illustrate the manner in which a tapered fiber tip can be used to collimate fiber output and further reduce retro-repulsion according to an embodiment of the present invention.

FIG. 3 is a flowchart illustrating a retro-repulsion reducing method according to a preferred embodiment of the invention.

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

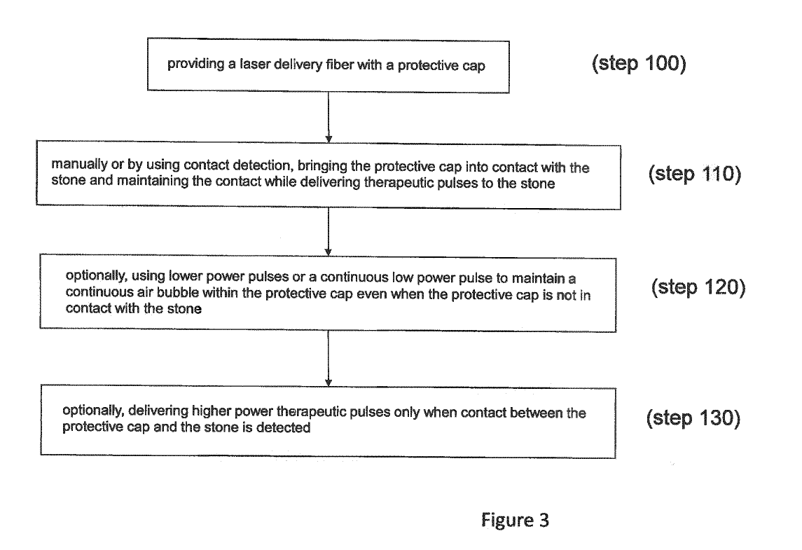

According to a preferred embodiment of the invention, as illustrated in FIG. 3, retro-repulsion during stone lithotripsy is reduced by the following steps:
(a) providing a laser delivery fiber with a protective cap of the type disclosed in copending PCT Publication No. PCT/US2017/031091, incorporated herein by reference, including any of the embodiments illustrated in FIGS. 3-12 of the copending PCT publication (step 100); and
(b) maintaining contact between the protective cap and the stone during delivery of therapeutic pulses to the stone (step 110).

In the preferred embodiment of the invention, the laser may be a pulsed Holmium laser with a pulse duration of greater than 250 micro seconds. The protective cap may be a generally cylindrical sleeve that extends beyond the fiber tip to maintain a minimum spacing between the fiber tip and the stone during lasing, in order to prevent contact and consequent fiber degradation, and may be made of a relatively soft, compressible material such as nylon, polyester, or Teflon™ that is fitted over a stripped section of the fiber core or cladding and held in place by welding or a compression fit, as disclosed in the copending PCT application. Alternatively, the preferred embodiment may be used with lasing apparatus other than pulsed Holmium lasers and protective caps other than soft caps, such as protective caps made of a harder materials such as glass, ceramic or metal. it will be appreciated that the term “cap” is not intended to be limited to a particular structure, and that the cap may be a cylindrical sleeve or have other configurations, so long as the cap extends beyond the fiber tip and is shaped to contact the stone in such a way as to limit the amount of water between the stone and the fiber tip.

In addition to the two method steps of the preferred embodiment set forth above, the method of the invention may optionally include the following steps:
(c) using lower power pulses or a continuous low power pulse to maintain a continuous air bubble within the protective cap (step 120); and
(d) delivering higher power therapeutic pulses only when the protective cap is in contact with the stone (step 130).

Step (d) may be implemented by using the proximity detection method and apparatus disclosed in copending U.S. Provisional Patent Appl. Ser. No. 62/513,791, which detects stone proximity to the fiber tip, and which can be used to provide a signal indicative of contact between the stone and the protective cap for the purpose of limiting firing of higher power therapeutic pulses unless the protective cap is in contact with the stone, as described in the copending Provisional U.S. Patent Appl. Ser. No. 62/513,791. It is also within the scope of the present invention to use proximity detection methods and apparatus other than the one disclosed in the copending provisional application.

Step (d) may alternatively be implemented manually, based on operator observation of stone position combined with the use of a lower power pulse or pulses to maintain a continuous air bubble or channel within the protective cap when the higher power therapeutic pulses are not being manually triggered by the operator upon detection of contact between the stone and the protective cap. Manual triggering can be achieved by foot pedal, a hand-operated controller, or any other manual control.

It will be appreciated that the term “therapeutic pulses” as used herein refers to higher power pulses intended to destroy or vaporize a stone.

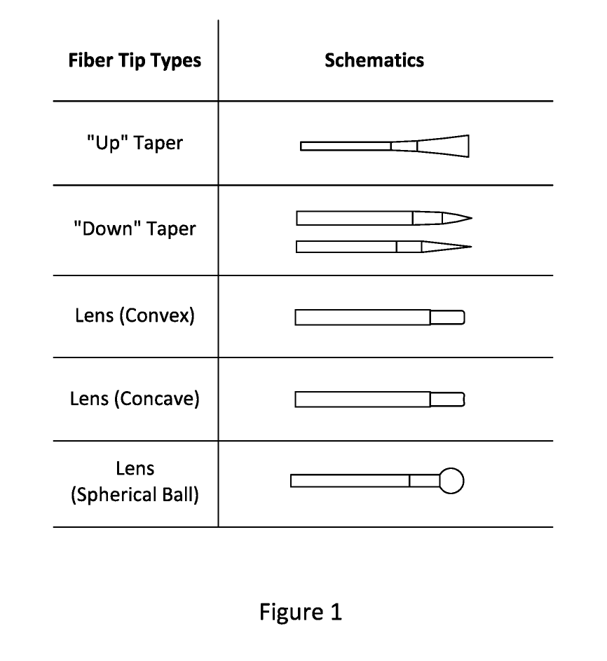

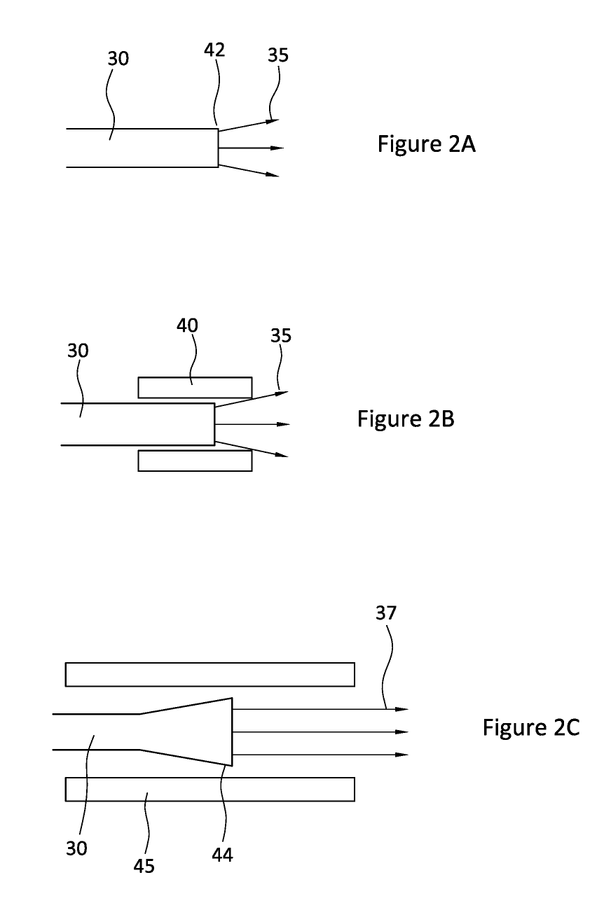

Referring to FIGS. 1 and 2A-2C, the fiber 30 may have a flat tip 42, shown in FIGS. 2A and 2B, an outwardly tapered tip 44 shown in FIG. 2C, or any of the other tip shapes shown in FIG. 1, including rounded, ball, concave, convex, and inwardly tapered shapes. The outwardly tapered tip shape shown in FIG. 2C has the particular advantage that, by choosing an appropriate taper angle, the laser pulse output 37 can be collimated or the numerical aperture lowered to allow the fiber tip 44 to be further recessed within the protective cap 46 and facilitate optimization of fiber position to minimize the pressure wave at the distal end of the protective cap and increase power density.

What is claimed is:

1. A method of reducing retro-repulsion during laser lithotripsy, comprising the steps of:
(a) providing a laser delivery fiber with a protective cap fitted onto an end of the laser delivery fiber and extending a predetermined distance beyond a tip of the fiber to prevent contact between the tip of the fiber and a stone; and
(b) maintaining contact between the protective cap and the stone during delivery of therapeutic pulses to the stone.

2. A retro-repulsion reducing method as claimed in claim 1, wherein the laser is a pulsed Holmium laser with a pulse duration of greater than 250 micro seconds.

3. A retro-repulsion reducing method as claimed in claim 1, wherein the protective cap may be a generally cylindrical sleeve that extends beyond the fiber tip to maintain a minimum spacing between the fiber tip and the stone during lasing.

4. A retro-repulsion reducing method as claimed in claim 3, wherein the protective cap is made of a soft, compressible material such as nylon, polyester, or Teflon™ that is fitted over a stripped section of the fiber core or cladding and held in place by welding or a compression fit.

5. A retro-repulsion reducing method as claimed in claim 1, further comprising the steps of:
(c) using lower power pulses or a continuous low power pulse to maintain a continuous air bubble within the protective cap; and
(d) delivering higher power therapeutic pulses only when the protective cap is in contact with the stone.

6. A retro-repulsion reducing method as claimed in claim 1, wherein step (d) is implemented by using a proximity detection method that detects stone proximity to the fiber tip, provides a signal indicative of contact between the stone and the protective cap, and limits firing of higher power therapeutic pulses unless the protective cap is in contact with the stone.

7. A retro-repulsion reducing method as claimed in claim 1, wherein step (d) is implemented manually, based on operator observation of stone position combined with the use of a lower power pulse or pulses to maintain a continuous air bubble or channel within the protective cap when the higher power therapeutic pulses are not being manually triggered by the operator upon detection of contact between the stone and the protective cap.

8. A retro-repulsion reducing method as claimed in claim 1, wherein the laser delivery fiber has a tip that is outwardly tapered to collimate a pulsed laser output beam or lower a numerical aperture and allow the fiber tip to be further recessed within the protective cap.
